SEQUENCE LISTING (96 sequences)

SEQ ID NO: 1 - RNA, 24 nt, synthetic construct
  misc_feature 1..24: note = Synthetic: bta.mir.146a
  source 1..24: mol_type = other RNA
tgagaactgaattccataggttgt

SEQ ID NO: 2 - RNA, 22 nt, synthetic construct
  misc_feature 1..22: note = Synthetic: bta.mir.433
  source 1..22: mol_type = other RNA
atcatgatgggctcctcggtgt

SEQ ID NO: 3 - RNA, 23 nt, synthetic construct
  misc_feature 1..23: note = Synthetic: bta.mir.29b
  source 1..23: mol_type = other RNA
tagcaccatttgaaatcagtgtt

SEQ ID NO: 4 - RNA, 23 nt, synthetic construct
  misc_feature 1..23: note = Synthetic: bta.mir.105a
  source 1..23: mol_type = other RNA
tcaaatgctcagactcctgtggt

SEQ ID NO: 5 - RNA, 22 nt, synthetic construct
  misc_feature 1..22: note = Synthetic: bta.mir.100
  source 1..22: mol_type = other RNA
aacccgtagatccgaacttgtg

SEQ ID NO: 6 - RNA, 22 nt, synthetic construct
  misc_feature 1..22: note = Synthetic: bta.mir.1247.5p
  source 1..22: mol_type = other RNA
acccgtcccgtgcgtccccgga

SEQ ID NO: 7 - RNA, 23 nt, synthetic construct
  misc_feature 1..23: note = Synthetic: bta.mir.155
  source 1..23: mol_type = other RNA
ttaatgctaatcgtgataggggt

SEQ ID NO: 8 - RNA, 23 nt, synthetic construct
  misc_feature 1..23: note = Synthetic: bta.mir.6517
  source 1..23: mol_type = other RNA
tcagggtccgtgagctcctcggc

SEQ ID NO: 9 - RNA, 23 nt, synthetic construct
  misc_feature 1..23: note = Synthetic: bta.mir.24
  source 1..23: mol_type = other RNA
gtgcctactgagctgatatcagt

SEQ ID NO: 10 - RNA, 22 nt, synthetic construct
  misc_feature 1..22: note = Synthetic: bta.mir.184
  source 1..22: mol_type = other RNA
tggacggagaactgataagggt

SEQ ID NO: 11 - RNA, 22 nt, synthetic construct
  misc_feature 1..22: note = Synthetic: bta.mir.142.3p
  source 1..22: mol_type = other RNA
agtgtttcctactttatggatg

SEQ ID NO: 12 - RNA, 23 nt, synthetic construct
  misc_feature 1..23: note = Synthetic: bta.mir.137
  source 1..23: mol_type = other RNA
ttattgcttaagaatacgcgtag

SEQ ID NO: 13 - RNA, 23 nt, synthetic construct
  misc_feature 1..23: note = Synthetic: bta.mir.582
  source 1..23: mol_type = other RNA
ttacagttgttcaaccagttact

SEQ ID NO: 14 - RNA, 23 nt, synthetic construct
  misc_feature 1..23: note = Synthetic: bta.mir.196b
  source 1..23: mol_type = other RNA
taggtagtttcctgttgttggga

SEQ ID NO: 15 - RNA, 23 nt, synthetic construct
  misc_feature 1..23: note = Synthetic: bta.mir.19b
  source 1..23: mol_type = other RNA
tgtgcaaatccatgcaaaactga

SEQ ID NO: 16 - RNA, 24 nt, synthetic construct
  misc_feature 1..24: note = Synthetic: bta.mir.21.5p
  source 1..24: mol_type = other RNA
tagcttatcagactgatgttgact

SEQ ID NO: 17 - RNA, 22 nt, synthetic construct
  misc_feature 1..22: note = Synthetic: bta.mir.133b
  source 1..22: mol_type = other RNA
tttggtccccttcaaccagcta

SEQ ID NO: 18 - RNA, 21 nt, synthetic construct
  misc_feature 1..21: note = Synthetic: bta.mir.378c
  source 1..21: mol_type = other RNA
actggacttggagtcagaagt

SEQ ID NO: 19 - RNA, 23 nt, synthetic construct
  misc_feature 1..23: note = Synthetic: bta.mir.32
  source 1..23: mol_type = other RNA
tattgcacatgactaagttgcat

SEQ ID NO: 20 - RNA, 21 nt, synthetic construct
  misc_feature 1..21: note = Synthetic: bta.mir.202
  source 1..21: mol_type = other RNA
ttcctatgcatatacttcttt

SEQ ID NO: 21 - RNA, 22 nt, synthetic construct
  misc_feature 1..22: note = Synthetic: bta.mir.1271
  source 1..22: mol_type = other RNA
cttggcacctagtaagtactca

SEQ ID NO: 22 - RNA, 22 nt, synthetic construct
  misc_feature 1..22: note = Synthetic: bta.mir.7857.5p
  source 1..22: mol_type = other RNA
atagccagttggggaagaatgc

SEQ ID NO: 23 - RNA, 23 nt, synthetic construct
  misc_feature 1..23: note = Synthetic: bta.mir.29a
  source 1..23: mol_type = other RNA
ctagcaccatctgaaatcggtta

SEQ ID NO: 24 - RNA, 25 nt, synthetic construct
  misc_feature 1..25: note = Synthetic: bta.miR.301a
  source 1..25: mol_type = other RNA
cagtgcaatagtattgtcaaagcat

SEQ ID NO: 25 - RNA, 22 nt, synthetic construct
  misc_feature 1..22: note = Synthetic: bta.mir.93
  source 1..22: mol_type = other RNA
caaagtgctgttcgtgcaggta

SEQ ID NO: 26 - RNA, 23 nt, synthetic construct
  misc_feature 1..23: note = Synthetic: bta.mir.20a.5p
  source 1..23: mol_type = other RNA
taaagtgcttatagtgcaggtag

SEQ ID NO: 27 - RNA, 22 nt, synthetic construct
  misc_feature 1..22: note = Synthetic: bta.mir.16a.5p
  source 1..22: mol_type = other RNA
tagcagcacgtaaatattggtg

SEQ ID NO: 28 - RNA, 24 nt, synthetic construct
  misc_feature 1..24: note = Synthetic: bta.mir.17.5p
  source 1..24: mol_type = other RNA
caaagtgcttacagtgcaggtagt

SEQ ID NO: 29 - RNA, 22 nt, synthetic construct
  misc_feature 1..22: note = Synthetic: bta.mir.92a
  source 1..22: mol_type = other RNA
tattgcacttgtcccggcctgt

SEQ ID NO: 30 - RNA, 23 nt, synthetic construct
  misc_feature 1..23: note = Synthetic: cel.mir.70.3p
  source 1..23: mol_type = other RNA
taatacgtcgttggtgtttccat

SEQ ID NO: 31 - RNA, 22 nt, synthetic construct
  misc_feature 1..22: note = Synthetic: oan.mir.7417.5p
  source 1..22: mol_type = other RNA
ttccccactctgagcacacagc

SEQ ID NO: 32 - RNA, 22 nt, synthetic construct
  misc_feature 1..22: note = Synthetic: ath.mir167d
  source 1..22: mol_type = other RNA
tgaagctgccagcatgatctgg

SEQ ID NO: 33 - DNA, 24 bp, synthetic construct
  misc_feature 1..24: note = Synthetic: bta.mir.146a
  source 1..24: mol_type = other DNA
tgagaactgaattccataggttgt

SEQ ID NO: 34 - DNA, 22 bp, synthetic construct
  misc_feature 1..22: note = Synthetic: bta.mir.433
  source 1..22: mol_type = other DNA
atcatgatgggctcctcggtgt

SEQ ID NO: 35 - DNA, 23 bp, synthetic construct
  misc_feature 1..23: note = Synthetic: bta.mir.29b
  source 1..23: mol_type = other DNA
tagcaccatttgaaatcagtgtt

SEQ ID NO: 36 - DNA, 23 bp, synthetic construct
  misc_feature 1..23: note = Synthetic: bta.mir.105a
  source 1..23: mol_type = other DNA
tcaaatgctcagactcctgtggt

SEQ ID NO: 37 - DNA, 22 bp, synthetic construct
  misc_feature 1..22: note = Synthetic: bta.mir.100
  source 1..22: mol_type = other DNA
aacccgtagatccgaacttgtg

SEQ ID NO: 38 - DNA, 22 bp, synthetic construct
  misc_feature 1..22: note = Synthetic: bta.mir.1247.5p
  source 1..22: mol_type = other DNA
acccgtcccgtgcgtccccgga

SEQ ID NO: 39 - DNA, 23 bp, synthetic construct
  misc_feature 1..23: note = Synthetic: bta.mir.155
  source 1..23: mol_type = other DNA
ttaatgctaatcgtgataggggt

SEQ ID NO: 40 - DNA, 23 bp, synthetic construct
  misc_feature 1..23: note = Synthetic: bta.mir.6517
  source 1..23: mol_type = other DNA
tcagggtccgtgagctcctcggc

SEQ ID NO: 41 - DNA, 23 bp, synthetic construct
  misc_feature 1..23: note = Synthetic: bta.mir.24
  source 1..23: mol_type = other DNA
gtgcctactgagctgatatcagt

SEQ ID NO: 42 - DNA, 22 bp, synthetic construct
  misc_feature 1..22: note = Synthetic: bta.mir.184
  source 1..22: mol_type = other DNA
tggacggagaactgataagggt

SEQ ID NO: 43 - DNA, 22 bp, synthetic construct
  misc_feature 1..22: note = Synthetic: bta.mir.142.3p
  source 1..22: mol_type = other DNA
agtgtttcctactttatggatg

SEQ ID NO: 44 - DNA, 23 bp, synthetic construct
  misc_feature 1..23: note = Synthetic: bta.mir.137
  source 1..23: mol_type = other DNA
ttattgcttaagaatacgcgtag

SEQ ID NO: 45 - DNA, 23 bp, synthetic construct
  misc_feature 1..23: note = Synthetic: bta.mir.582
  source 1..23: mol_type = other DNA
ttacagttgttcaaccagttact

SEQ ID NO: 46 - DNA, 23 bp, synthetic construct
  misc_feature 1..23: note = Synthetic: bta.mir.196b
  source 1..23: mol_type = other DNA
taggtagtttcctgttgttggga

SEQ ID NO: 47 - DNA, 23 bp, synthetic construct
  misc_feature 1..23: note = Synthetic: bta.mir.19b
  source 1..23: mol_type = other DNA
tgtgcaaatccatgcaaaactga

SEQ ID NO: 48 - DNA, 24 bp, synthetic construct
  misc_feature 1..24: note = Synthetic: bta.mir.21.5p
  source 1..24: mol_type = other DNA
tagcttatcagactgatgttgact

SEQ ID NO: 49 - DNA, 22 bp, synthetic construct
  misc_feature 1..22: note = Synthetic: bta.mir.133b
  source 1..22: mol_type = other DNA
tttggtccccttcaaccagcta

SEQ ID NO: 50 - DNA, 21 bp, synthetic construct
  misc_feature 1..21: note = Synthetic: bta.mir.378c
  source 1..21: mol_type = other DNA
actggacttggagtcagaagt

SEQ ID NO: 51 - DNA, 23 bp, synthetic construct
  misc_feature 1..23: note = Synthetic: bta.mir.32
  source 1..23: mol_type = other DNA
tattgcacatgactaagttgcat

SEQ ID NO: 52 - DNA, 21 bp, synthetic construct
  misc_feature 1..21: note = Synthetic: bta.mir.202
  source 1..21: mol_type = other DNA
ttcctatgcatatacttcttt

SEQ ID NO: 53 - DNA, 22 bp, synthetic construct
  misc_feature 1..22: note = Synthetic: bta.mir.1271
  source 1..22: mol_type = other DNA
cttggcacctagtaagtactca

SEQ ID NO: 54 - DNA, 22 bp, synthetic construct
  misc_feature 1..22: note = Synthetic: bta.mir.7857.5p
  source 1..22: mol_type = other DNA
atagccagttggggaagaatgc

SEQ ID NO: 55 - DNA, 23 bp, synthetic construct
  misc_feature 1..23: note = Synthetic: bta.mir.29a
  source 1..23: mol_type = other DNA
ctagcaccatctgaaatcggtta

SEQ ID NO: 56 - DNA, 25 bp, synthetic construct
  misc_feature 1..25: note = Synthetic: bta.miR.301a
  source 1..25: mol_type = other DNA
cagtgcaatagtattgtcaaagcat

SEQ ID NO: 57 - DNA, 22 bp, synthetic construct
  misc_feature 1..22: note = Synthetic: bta.mir.93
  source 1..22: mol_type = other DNA
caaagtgctgttcgtgcaggta

SEQ ID NO: 58 - DNA, 23 bp, synthetic construct
  misc_feature 1..23: note = Synthetic: bta.mir.20a.5p
  source 1..23: mol_type = other DNA
taaagtgcttatagtgcaggtag

SEQ ID NO: 59 - DNA, 22 bp, synthetic construct
  misc_feature 1..22: note = Synthetic: bta.mir.16a.5p
  source 1..22: mol_type = other DNA
tagcagcacgtaaatattggtg

SEQ ID NO: 60 - DNA, 24 bp, synthetic construct
  misc_feature 1..24: note = Synthetic: bta.mir.17.5p
  source 1..24: mol_type = other DNA
caaagtgcttacagtgcaggtagt

SEQ ID NO: 61 - DNA, 22 bp, synthetic construct
  misc_feature 1..22: note = Synthetic: bta.mir.92a
  source 1..22: mol_type = other DNA
tattgcacttgtcccggcctgt

SEQ ID NO: 62 - DNA, 23 bp, synthetic construct
  misc_feature 1..23: note = Synthetic: cel.mir.70.3p
  source 1..23: mol_type = other DNA
taatacgtcgttggtgtttccat

SEQ ID NO: 63 - DNA, 22 bp, synthetic construct
  misc_feature 1..22: note = Synthetic: oan.mir.7417.5p
  source 1..22: mol_type = other DNA
ttccccactctgagcacacagc

SEQ ID NO: 64 - DNA, 22 bp, synthetic construct
  misc_feature 1..22: note = Synthetic: ath.mir167d
  source 1..22: mol_type = other DNA
tgaagctgccagcatgatctgg

SEQ ID NO: 65 - DNA, 24 bp, synthetic construct
  misc_feature 1..24: note = Synthetic: bta.mir.146a
  source 1..24: mol_type = other DNA
acaacctatggaattcagttctca

SEQ ID NO: 66 - DNA, 22 bp, synthetic construct
  misc_feature 1..22: note = Synthetic: bta.mir.433
  source 1..22: mol_type = other DNA
acaccgaggagcccatcatgat

SEQ ID NO: 67 - DNA, 23 bp, synthetic construct
  misc_feature 1..23: note = Synthetic: bta.mir.29b
  source 1..23: mol_type = other DNA
aacactgatttcaaatggtgcta

SEQ ID NO: 68 - DNA, 23 bp, synthetic construct
  misc_feature 1..23: note = Synthetic: bta.mir.105a
  source 1..23: mol_type = other DNA
accacaggagtctgagcatttga

SEQ ID NO: 69 - DNA, 22 bp, synthetic construct
  misc_feature 1..22: note = Synthetic: bta.mir.100
  source 1..22: mol_type = other DNA
cacaagttcggatctacgggtt

SEQ ID NO: 70 - DNA, 22 bp, synthetic construct
  misc_feature 1..22: note = Synthetic: bta.mir.1247.5p
  source 1..22: mol_type = other DNA
tccggggacgcacgggacgggt

SEQ ID NO: 71 - DNA, 23 bp, synthetic construct
  misc_feature 1..23: note = Synthetic: bta.mir.155
  source 1..23: mol_type = other DNA
acccctatcacgattagcattaa

SEQ ID NO: 72 - DNA, 23 bp, synthetic construct
  misc_feature 1..23: note = Synthetic: bta.mir.6517
  source 1..23: mol_type = other DNA
gccgaggagctcacggaccctga

SEQ ID NO: 73 - DNA, 23 bp, synthetic construct
  misc_feature 1..23: note = Synthetic: bta.mir.24
  source 1..23: mol_type = other DNA
actgatatcagctcagtaggcac

SEQ ID NO: 74 - DNA, 22 bp, synthetic construct
  misc_feature 1..22: note = Synthetic: bta.mir.184
  source 1..22: mol_type = other DNA
acccttatcagttctccgtcca

SEQ ID NO: 75 - DNA, 22 bp, synthetic construct
  misc_feature 1..22: note = Synthetic: bta.mir.142.3p
  source 1..22: mol_type = other DNA
catccataaagtaggaaacact

SEQ ID NO: 76 - DNA, 23 bp, synthetic construct
  misc_feature 1..23: note = Synthetic: bta.mir.137
  source 1..23: mol_type = other DNA
ctacgcgtattcttaagcaataa

SEQ ID NO: 77 - DNA, 23 bp, synthetic construct
  misc_feature 1..23: note = Synthetic: bta.mir.582
  source 1..23: mol_type = other DNA
agtaactggttgaacaactgtaa

SEQ ID NO: 78 - DNA, 23 bp, synthetic construct
  misc_feature 1..23: note = Synthetic: bta.mir.196b
  source 1..23: mol_type = other DNA
tcccaacaacaggaaactaccta

SEQ ID NO: 79 - DNA, 23 bp, synthetic construct
  misc_feature 1..23: note = Synthetic: bta.mir.19b
  source 1..23: mol_type = other DNA
tcagttttgcatggatttgcaca

SEQ ID NO: 80 - DNA, 24 bp, synthetic construct
  misc_feature 1..24: note = Synthetic: bta.mir.21.5p
  source 1..24: mol_type = other DNA
agtcaacatcagtctgataagcta

SEQ ID NO: 81 - DNA, 22 bp, synthetic construct
  misc_feature 1..22: note = Synthetic: bta.mir.133b
  source 1..22: mol_type = other DNA
tagctggttgaaggggaccaaa

SEQ ID NO: 82 - DNA, 21 bp, synthetic construct
  misc_feature 1..21: note = Synthetic: bta.mir.378c
  source 1..21: mol_type = other DNA
acttctgactccaagtccagt

SEQ ID NO: 83 - DNA, 23 bp, synthetic construct
  misc_feature 1..23: note = Synthetic: bta.mir.32
  source 1..23: mol_type = other DNA
atgcaacttagtcatgtgcaata

SEQ ID NO: 84 - DNA, 21 bp, synthetic construct
  misc_feature 1..21: note = Synthetic: bta.mir.202
  source 1..21: mol_type = other DNA
aaagaagtatatgcataggaa

SEQ ID NO: 85 - DNA, 22 bp, synthetic construct
  misc_feature 1..22: note = Synthetic: bta.mir.1271
  source 1..22: mol_type = other DNA
tgagtacttactaggtgccaag

SEQ ID NO: 86 - DNA, 22 bp, synthetic construct
  misc_feature 1..22: note = Synthetic: bta.mir.7857.5p
  source 1..22: mol_type = other DNA
gcattcttccccaactggctat

SEQ ID NO: 87 - DNA, 23 bp, synthetic construct
  misc_feature 1..23: note = Synthetic: bta.mir.29a
  source 1..23: mol_type = other DNA
taaccgatttcagatggtgctag

SEQ ID NO: 88 - DNA, 25 bp, synthetic construct
  misc_feature 1..25: note = Synthetic: bta.miR.301a
  source 1..25: mol_type = other DNA
atgctttgacaatactattgcactg

SEQ ID NO: 89 - DNA, 22 bp, synthetic construct
  misc_feature 1..22: note = Synthetic: bta.mir.93
  source 1..22: mol_type = other DNA
tacctgcacgaacagcactttg

SEQ ID NO: 90 - DNA, 23 bp, synthetic construct
  misc_feature 1..23: note = Synthetic: bta.mir.20a.5p
  source 1..23: mol_type = other DNA
ctacctgcactataagcacttta

SEQ ID NO: 91 - DNA, 22 bp, synthetic construct
  misc_feature 1..22: note = Synthetic: bta.mir.16a.5p
  source 1..22: mol_type = other DNA
caccaatatttacgtgctgcta

SEQ ID NO: 92 - DNA, 24 bp, synthetic construct
  misc_feature 1..24: note = Synthetic: bta.mir.17.5p
  source 1..24: mol_type = other DNA
actacctgcactgtaagcactttg

SEQ ID NO: 93 - DNA, 22 bp, synthetic construct
  misc_feature 1..22: note = Synthetic: bta.mir.92a
  source 1..22: mol_type = other DNA
acaggccgggacaagtgcaata

SEQ ID NO: 94 - DNA, 23 bp, synthetic construct
  misc_feature 1..23: note = Synthetic: cel.mir.70.3p
  source 1..23: mol_type = other DNA
atggaaacaccaacgacgtatta

SEQ ID NO: 95 - DNA, 22 bp, synthetic construct
  misc_feature 1..22: note = Synthetic: oan.mir.7417.5p
  source 1..22: mol_type = other DNA
gctgtgtgctcagagtggggaa

SEQ ID NO: 96 - DNA, 22 bp, synthetic construct
  misc_feature 1..22: note = Synthetic: ath.mir167d
  source 1..22: mol_type = other DNA
ccagatcatgctggcagcttca

(19) United States
(12) Patent Application Publication
(10) Pub. No.: US 2025/0166826 A1
(43) Pub. Date: May 22, 2025

(54) RAPID ASSAY FOR DETECTING MIRNA: COMPOSITIONS AND METHODS OF USE

(71) Applicant: MI:RNA LTD., Edinburgh (GB)

(72) Inventors: Eve HANKS, Edinburgh (GB); Robert COULTOUS, Whitburn (GB)

(73) Assignee: MI:RNA LTD., Edinburgh (GB)

(21) Appl. No.: 18/949,011

(22) Filed: Nov. 15, 2024

Related U.S. Application Data
(60) Provisional application No. 63/599,860, filed on Nov. 16, 2023.

(51) Int. Cl. G16H 50/20; C12N 15/10; C12Q 1/6806; C12Q 1/689

(52) U.S. Cl. CPC G16H 50/20; C12N 15/1096; C12Q 1/6806; C12Q 1/689; C12Q 2600/178

(57) ABSTRACT

An diagnostic assay for rapidly detecting level of expression of mRNA within low volume or low expression samples by proxy of generated cDNA. The method provides for the detection of cDNA of a diseased subject with at least one pre-determined reference level cDNA characteristic of a non-diseased subject wherein a deviation of the level of expression of said cDNA in comparison with the at least one reference level cDNA allows for the diagnosis and/or prognosis of the disease.

### CROSS-REFERENCE TO RELATED APPLICATIONS

This invention claims the benefit of priority to U.S. Provisional Application No. 63/599,860, filed on Nov. 16, 2023, the entire contents of which are incorporated herein by reference.

### SEQUENCE LISTING

The application contains a Sequence Listing which has been submitted electronically in .XML format and is hereby incorporated by reference in its entirety. Said .XML copy, created on Nov. 13, 2024, is named “068075.004US1 Seq Listing.xml” and is 122,876 bytes in size. The sequence listing contained in this .XML file is part of the specification and is hereby incorporated by reference herein in its entirety.

### FIELD

This invention relates generally to methods of miRNA detection by proxy of generated cDNA.

### BACKGROUND

Recent advances in genomics, proteomics, cellomics and metabolomics have provided us with large libraries of biological molecules and chemical compounds that modulate various biological processes. Such developments have necessitated the need for high throughput analysis/screening where millions of biochemical, genetic or pharmacological assays are performed and analyzed in a parallel fashion to find active compounds against biological targets. In addition, the analysis, detection, identification and quantification of these markers provide powerful new means to study biology and pathology and to develop new diagnostics and therapeutics.

Many biological and disease markers exist at low concentrations in biological samples, yet play important roles in biological and pathological processes. The ability to rapidly and selectively detect low abundance is critically important to elucidate new biology, to monitor, detect a disease or disorder, and to monitor therapeutic responses and to develop new therapeutics.

Thus, the invention presented herein provides methods of detecting miRNA within low volume or low expression samples by proxy of generated cDNA. Compared to the direct detection of miRNA within an extracted sample, the additional step of cDNA synthesis provides an amplification stage, increasing sensitivity of detection in low volume or low expression samples.

### SUMMARY

In accordance with the purpose(s) of this invention, as embodied and broadly described herein, this invention, in one aspect, relates to a method for detecting the presence of mycobacterial disease in a subject, comprising the steps of: isolating miRNA molecules within a sample from a subject; translating the miRNA molecules to cDNA molecules; amplifying the cDNA molecules to a detectable concentration; probing for the cDNA molecules complimentary to the desired miRNA markers; determining a level of expression of the miRNA molecules within a sample from a subject by the level of cDNA molecules probed for the desired miRNA markers; and using one or more Artificial Intelligence (AI) model to predict the disease condition of the subject.

In one embodiment, the one or more AI model compares the level of expression of each cDNA molecule with at least one pre-determined reference level cDNA molecule characteristic of a non-diseased subject wherein a deviation of the level of expression of said cDNA molecule in comparison with the at least one reference level cDNA molecule allows for the diagnosis and/or prognosis of the disease.

In another embodiment, the miRNA molecules are selected from a group consisting of nucleic acid sequence having at least 95%, 97%, 98% or 99% sequence identity to SEQ ID NO: 1, 2, 3, 4, 5, 6, 7, 8, 9, 10, 11, 12, 13, 14, 15, 16, 17, 18, 19, 20, 21, 22, 23 and 24. In other embodiments, the cDNA molecules are selected from a group consisting of nucleic acid sequence having at least 95%, 97%, 98% or 99% sequence identity to SEQ ID NO:33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, and 88.

In another embodiment, the method further comprises the use of at least one normalizer and/or control miRNA or cDNA molecule. In one embodiment, at least one normalizer mRNA molecule is selected from a group consisting of nucleic acid sequence having at least 95%, 97%, 98% or 99% sequence identity to SEQ ID NO:25, 26, 27, 28 and 29 and at least one normalizer cDNA molecule is selected from a group consisting of nucleic acid sequence having at least 95%, 97%, 98% or 99% sequence identity to SEQ ID NO:57, 58, 59, 60, 61, 89, 90, 91, 92, and 93. In one other embodiment, at least one control miRNA is selected from a group consisting of nucleic acid sequence having at least 95%, 97%, 98% or 99% sequence identity to SEQ ID NO:30, 31, and 32, and at least one control cDNA is selected from a group consisting of nucleic acid sequence having at least 95%, 97%, 98% or 99% sequence identity to SEQ ID NO:62, 63, 64, 94, 95 and 96.

In another embodiment, the method further comprises the step of using a machine learning algorithm for predictive modelling, wherein the method comprises the use of a combination of AI models.

In other embodiments, the subject is a mammal, wherein the subject is a cow, sheep, goat, deer, llama, alpaca or vicuna. In one other embodiment, the subject's disease is selected from a group consisting of mycobacterial disease, including but not restricted to Johne's disease, tuberculosis and Crohn's disease, or any other disease caused by Mycobacterium avium subspecies paratuberculosis (MAP) or any other species within the Mycobacterium genus. In additional embodiment, the sample obtained from the subject is a biofluid selected from the group consisting of blood, urine, milk, tissue fluid, saliva, milk, cerebrospinal fluid (CSF), faeces or another biofluid. In additional embodiments, the miRNA molecules obtained from the samples are cell free miRNA molecules.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTIONS OF THE DRAWINGS

The accompanying drawings, which are incorporated in and constitute a part of this specification, illustrate (one) several embodiment(s) of the invention and together with the description, serve to explain the principles of the invention.

FIGS. 1A-IC are bar graphs showing the relative expression of miRNA markers.

FIGS. 2A-2C are bar graphs showing the relative expression of miRNA markers adjusted for NTC MFI cut-off.

FIG. 3 shows a PCA of data points to show grouping.

### DETAILED DESCRIPTION

The present invention may be understood more readily by reference to the following detailed description of preferred embodiments of the invention and the Examples included therein and to the Figures and their previous and following description.

### I. Definitions

To facilitate an understanding of the principles and features of the various embodiments of the disclosure, various illustrative embodiments are explained herein. Although exemplary embodiments of the disclosure are explained in detail, it is to be understood that other embodiments are contemplated. Accordingly, it is not intended that the disclosure is limited in its scope to the details of construction and arrangement of components set forth in the description or examples. The disclosure is capable of other embodiments and of being practiced or carried out in various ways.

In describing the exemplary embodiments, specific terminology will be resorted to for the sake of clarity. As used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

The phrase “nucleic acid” or “polynucleotide sequence” refers to a single or double-stranded polymer of deoxyribonucleotide or ribonucleotide bases read from the 5′ to the 3′ end. Nucleic acids may also include modified nucleotides that permit correct read-through by a polymerase and do not alter expression of a polypeptide encoded by that nucleic acid.

A “coding sequence” or “coding region” refers to a nucleic acid molecule having sequence information necessary to produce a gene product, when the sequence is expressed.

A “probe” is defined as a nucleic acid capable of binding to a target nucleic acid of complementary sequence through one or more types of chemical bonds, usually through complementary base pairing, usually through hydrogen bond formation. A probe may include natural (i.e., A, G, C, T or U) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. Probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

As used herein, the term “microRNA” or “miRNA” or “miR” designates a non-coding RNA molecule having a length of about 17 to 25 nucleotides, specifically having a length of 17, 18, 19, 20, 21, 22, 23, 24 or 25 nucleotides which hybridizes to and regulates the expression of a coding messenger RNA.

The term “miRNA molecule” refers to any nucleic acid molecule representing the miRNA, including natural miRNA molecules, i.e. the mature miRNA, pre-miRNA, pri-miRNA.

As used herein, the term “cDNA” (complementary DNA) refers to a piece of DNA lacking internal, non-coding segments (introns) and regulatory sequences that determine transcription. cDNA is synthesized in the laboratory by reverse transcription from messenger RNA extracted from cells. The term cDNA may also be used to refer to reverse compliment cDNA.

Complementarity and percentage complementarity: Molecules with complementary nucleic acids form a stable duplex or triplex when the strands bind, (hybridize), to each other by forming Watson-Crick, Hoogsteen or reverse Hoogsteen base pairs. Stable binding occurs when an oligonucleotide remains detectably bound to a target nucleic acid sequence under the required conditions.

Complementarity is the degree to which bases in one nucleic acid strand base pair with the bases in a second nucleic acid strand. Complementarity is conveniently described by percentage, i.e. the proportion of nucleotides that form base pairs between two strands or within a specific region or domain of two strands. For example, if 10 nucleotides of a 15-nucleotide oligonucleotide form base pairs with a targeted region of a DNA molecule, that oligonucleotide is said to have 66.67% complementarity to the region of DNA targeted.

In the present disclosure, “sufficient complementarity” means that a sufficient number of base pairs exist between the oligonucleotide and the target sequence to achieve detectable binding, and in the case of the binding of an antigen, disrupt expression of gene products (such as adenosine receptors). When expressed or measured by percentage of base pairs formed, the percentage complementarity that fulfills this goal can range from as little as about 50% complementarity to full, (100%) complementary. In general, sufficient complementarity is at least about 50%, for example at least 75%, 90%, 95%, 98% or even 100% complementarity.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated nucleic acid of the present invention is separated from open reading frames that flank the desired gene and encode proteins other than the desired protein. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “sample” generally refers to tissue or organ sample, blood, cell-free blood such as serum and plasma, urine, saliva, milk and cerebrospinal fluid sample.

As used herein, the term “blood sample” refers to serum, plasma, cell-free blood, whole blood and its components, blood derived products or preparations. Plasma and serum are very useful as shown in the examples. Specifically, the blood sample is a cell-free blood sample.

The term “quantifying” or “quantification” as used herein refers to absolute quantification, i.e. determining the amount of the respective miRNA but also encompasses measuring the level of the respective miRNA and comparing said level with reference or control miRNA, or comparative expression to other quantified miRNA. Quantification of the respective miRNA as listed in the tables herein allow expression profiling of samples and thus allow identification of signatures associated with diseased samples, as well as identification of signatures associated with prognosis and response to treatment. The quantity of miRNAs or difference in miRNA levels can be determined by any of the methods described herein.

A “control”, “control sample”, or “reference value” or “reference level” are terms which can be used interchangeably herein, and are to be understood as a sample or standard used for comparison with the experimental sample.

The practice of the present invention employs, unless otherwise indicated, conventional techniques of molecular biology (including recombinant techniques), microbiology, cell biology, biochemistry, immunology, protein kinetics, and mass spectroscopy, which are within the skill of art. Such techniques are explained fully in the literature, such as Sambrook et al., 2000, Molecular Cloning: A Laboratory Manual, third edition, Cold Spring Harbor Laboratory Press; Current Protocols in Molecular Biology Volumes 1-3, John Wiley & Sons, Inc.; Kriegler, 1990, Gene Transfer and Expression: A Laboratory Manual, Stockton Press, New York; Dieffenbach et al., 1995, PCR Primer: A Laboratory Manual, Cold Spring Harbor Laboratory Press, each of which is incorporated herein by reference in its entirety. Procedures employing commercially available assay kits and reagents typically are used according to manufacturer-defined protocols unless otherwise noted.

Generally, the nomenclature and the laboratory procedures in recombinant DNA technology described below are those well-known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications.

Nucleotide sequences of mature miRNAs and their respective precursors are known in the art and available from the database miRBase or from Sanger database.

All of the specified miRNAs used according to the invention also encompass isoforms and variants thereof. For the purpose of the invention, the terms “isoforms and variants” (which have also be termed “isomirs”) of a reference miRNA include trimming variants (5′ trimming variants in which the 5′ dicing site is upstream or downstream from the reference miRNA sequence; 3′ trimming variants: the 3′ dicing site is upstream or downstream from the reference miRNA sequence), or variants having one or more nucleotide modifications (3′ nucleotide addition to the 3′ end of the reference miRNA; nucleotide substitution by changing nucleotides from the miRNA precursor), or the complementary mature microRNA strand including its isoforms and variants (for example for a given 5′ mature microRNA the complementary 3′ mature microRNA and vice-versa). With regard to nucleotide modification, the nucleotides relevant for RNA/RNA binding, i.e. the 5′-seed region and nucleotides at the cleavage/anchor side are excluded from modification.

In the following, if not otherwise stated, the term “miRNA” encompasses 3p and 5p strands and also its isoforms and variants.

### II. Compositions

The present invention provides genomic identifiers that can be used as target nucleic acid sequences for diagnosis of mycobacterial infection. The diagnostic targets can be used for identification the presence of Johne's disease or any other mycobacterial disease in a sample. Additionally, the invention can be used to diagnose any other disease which causes the dysregulation of the patient's miRNA expression profile, detectable by measuring of that patient's miRNA expression profile, or any disease which results in a detectable presence of pathogen miRNA.

The practice of the present invention employs, unless otherwise indicated, conventional techniques of molecular biology (including recombinant techniques), microbiology, cell biology, biochemistry, immunology, protein kinetics, and mass spectroscopy, which are within the skill of art. Such techniques are explained fully in the literature, such as Sambrook et al., 2000, Molecular Cloning: A Laboratory Manual, third edition, Cold Spring Harbor Laboratory Press; Current Protocols in Molecular Biology Volumes 1-3, John Wiley & Sons, Inc.; Kriegler, 1990, Gene Transfer and Expression: A Laboratory Manual, Stockton Press, New York; Dieffenbach et al., 1995, PCR Primer: A Laboratory Manual, Cold Spring Harbor Laboratory Press, each of which is incorporated herein by reference in its entirety. Procedures employing commercially available assay kits and reagents typically are used according to manufacturer-defined protocols unless otherwise noted.

Generally, the nomenclature and the laboratory procedures in recombinant DNA technology described below are those well-known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications.

Provided herein is a method for detecting the presence of a disease condition in a subject, comprising the steps of: (a) determining the level of expression of each of a plurality of miRNAs within a sample from a subject; and (b) using one or more Artificial Intelligence (AI) model to predict the disease condition of the subject.

A. miRNAs

In recent years, the study of the non-coding class of RNA termed microRNA (miRNA) has grown significantly because of their role in post-transcriptional gene regulation. The identification of novel miRNA sequences has often involved computational approaches, with validation by Northern blot analysis or microarray analysis. Traditional RT-PCR approaches are difficult to implement for mature miRNA detection because the approximately 22 nucleotide sequences are not of sufficient length for primer extension by the reverse transcriptase followed by detection with PCR primers of traditional design.

miRNAs are a class of non-coding RNA sequences that range in length from 17 to 24 nucleotides (nt). Mature miRNA sequences result from a two-step processing of pri-miRNA transcripts by Drosha to produce the pre-miRNA intermediate, followed by Dicer to form the mature miRNA. In the mature form, the miRNA binds to the 3′-untranslated region (UTR) of mRNA targets to form an RNAi-induced silencing complex (RISC), which can inhibit translation by a number of methods. miRNAs have been linked to several diverse functions, including developmental timing, as well as a number of diseases including cancer.

Several miRNA are only expressed in specific developmental stages or in specific cells. Exemplary embodiments of the present invention relate to a subset of miRNA sequences, whose expression levels are found to vary between normal and Mycobacterium infected cells, and the development of a system for monitoring their expression. The development of a system for monitoring miRNA expression levels can allow for a better understanding of their biological roles and thereby their potential role in Mycobacterium infections or other diseases or disorders. The correlation between miRNA expression data and its link to disease state in the body may ultimately play a key role in early diagnosis, identifying potential therapeutic targets, development of a treatment plan, monitoring of treatment, and prognosis. While the examples given herein describe a subset of miRNA, detection reagents for any noncoding RNA, including other miRNA is also contemplated.

In one aspect, the invention provides a method of detecting microRNA (miRNA) molecules that are present in a sample. Conversely, the method is a method of determining the absence of a miRNA of interest. The method generally comprises: optionally implementing a primer design protocol; optionally providing a sample containing or suspected of containing a miRNA of interest; adding a polyA tail to the 3′ end of the miRNA in the sample if the miRNA is present; combining i) an adapter primer (e.g., a reverse transcription oligonucleotide) that can anneal to the polyA tail, and ii) the sample that might contain the miRNA with an attached polyA tail, resulting in a mixture; exposing the mixture to conditions that permit reverse transcription with a primer to form a cDNA; and detecting the presence or absence of the cDNA, where detection comprises amplification of the cDNA.

Providing, whether it be in reference to primer oligonucleotides, a sample, or any other substance used in the method, can be any act that results in a particular substance being present in a particular environment. Broadly speaking, it can be any action that results in the practitioner obtaining and having in possession the substance of interest in a form suitable for use in the present method (the term “assay” being used herein interchangeable on occasion). Those of skill in the art are aware of numerous actions that can achieve this result. In addition, non-limiting examples are mentioned throughout this disclosure. For example, providing can be adding a substance to another substance to create a composition. It can include mixing two or more substances together to create a composition or mixture. It can also include isolating a substance or composition from its natural environment or the environment from which it came. Providing likewise can include obtaining a substance or composition in a purified or partially purified form from a supplier or vendor. Additionally, providing can include obtaining a sample suspected of containing a miRNA of interest, removing a portion for use in the present method, and maintaining the remaining amount of sample in a separate container from the portion to be used in the present method. Other examples are numerous and will be apparent to those of skill in the art.

Combining substances or compositions in the method means bringing two or more substances, compositions, components, etc. into contact such that a single composition of the two results. Any act that provides such a result is encompassed by this term, and those of skill in the art are aware of numerous ways to achieve the result. A non-limiting example of actions that are considered combining is adding a composition comprising one or more primer oligonucleotides to an aqueous sample containing or suspected of containing a miRNA species. Combining can also include actions that result in the combination being a homogeneous or otherwise mixed composition in which substances of one starting material are interspersed with substances from one or more other starting materials. Thus, combining can include mixing to make a mixture. It can therefore include stirring, repetitive pipetting of the combination, inverting a container containing the combination, shaking the combination, vortexing the combination, or even permitting the combination to stand for a sufficient amount of time for random diffusion to effect partial or complete mixing. Mixing can also include any action that might be required to maintain a homogeneous or nearly homogeneous composition, including, but not limited to performing a new action or repeating one or more actions that resulted in an initial mixture. In a homogeneous assay, poly(A) polymerase, reverse transcription, and PCR reaction components and RNA sample can be combined in a single tube and incubated under conditions which allows for quantitation of a miRNA. In this method, it may be desirable to prevent alteration of the RT adapter and/or PCR primers by the poly(A) polymerase. A potential method to prevent this alteration is to use the CleanAmp™ Primers for Improved Hot Start PCR (TriLink Biotechnologies). In these primers, the 3′ end is chemically blocked and is not extended until heated at 95° C. for 5-10 minutes.

In situations where the target miRNA is not present in the sample, the polyA tail will not be attached to a target miRNA, the cDNA of the target miRNA will not be made by reverse transcription and no signal or only background signal levels are detected after PCR amplification. Accordingly, in embodiments, the method is a method of determining the absence of a miRNA of interest in a sample. Of course, it is difficult to determine the veracity of a conclusion where the results are negative; however, though use of proper control reactions at one or more points in the assay, a conclusion of lack of miRNA can be drawn with high confidence. As discussed herein, one or more control reactions (positive and/or negative) can be included at each step in the assay to verify proper functioning of reagents and reaction components. Those of skill in the art are well qualified to implement appropriate control reactions without the need for each to be detailed herein.

In embodiments, a cDNA is created by reverse transcription, however, the miRNA-specific primer does not anneal to the cDNA. By does not anneal, it is meant that the amount of annealing that occurs is undetectable or not significantly different than the amount that can be detected in a composition that lacks the miRNA cDNA, but is otherwise identical. The lack of annealing is typically determined by assaying for the presence or absence of an amplification product generated with the miRNA-specific primer. In other embodiments, there is enough complementarity between the miRNA primer and the mRNA target so that annealing occurs between the miRNA-specific primer and the mRNA target during PCR amplification. In this case, signal may be seen. However, the presence of the target miRNA in the sample significantly increases the amount of annealing of the miRNA-specific forward primer to the cDNA above the level that would occur in the absence of the target miRNA. Accordingly, the method of the invention is capable of detecting the presence or absence of a specific miRNA, and can preferably distinguish between miRNA presence and the mere presence of mRNA comprising a similar target sequence.

The method of the invention is also capable of detecting the presence or absence of more than one miRNA species. In other words, although the primers are designed to be specific, the term specific does not imply a one-to-one specificity. Rather, it encompasses binding to all nucleotide sequences that are complementary, at least over a sufficient length to permit hybridization under the conditions used. As the invention is most useful for specific detection of miRNA, typically the hybridization conditions will be set such that complementarity is very high, on the order of 90% or greater over a 10 nucleotide window of contiguous nucleotides. In another example, the complementarity is not as high and can be as low as about 60% over a 10 nucleotide window of contiguous nucleotides. In other embodiments, the complementarity of the primer to the miRNA species may be 60% over less than a 10 nucleotide window, such as a 8 nucleotide window or 60% over more than a 10 nucleotide window.

In embodiments, the method can detect fewer than or about 60 to more than or about 600 different miRNA species in one RNA sample. The high number of different miRNA species that are detected in one RNA sample is because only a small portion of the RT reaction is used as a miRNA template in separate PCR using different miRNA-specific primers.

The method of the present invention differs from other methods that use a miRNA-specific primer for reverse transcription. For example, in the present invention, a universal primer is used for reverse transcription, which results in cDNA being made from all of the miRNA and mRNA species present. Unlike other methods, one or multiple miRNA-specific primers can then be employed in the PCR amplification step to detect a specific miRNA (or mRNA) or a number of different species of mRNA or miRNA in the sample. The method may also be used to detect the mRNA target and the corresponding miRNA in the same sample.

Nucleotide sequences of mature miRNAs and their respective precursors are known in the art and available from the database miRBase or from Sanger database.

Identical polynucleotides as used herein in the context of a polynucleotide to be detected by the method as described herein may have a nucleic acid sequence with an identity of at least 90%, 95%, 97%, 98% or 99% or less than 3 or 2 single nucleotide modifications compared to a polynucleotide.

### III. Methods of Detection

The method of the invention comprises detecting, often indirectly, the presence of a reverse transcriptase reaction product, such as a cDNA. The cDNA may be one produced from pri-miRNA, miRNA, or mRNA. In one embodiment, the invention provides methods of determining a level of expression of the miRNA molecules within a sample from a subject by the level of cDNA or complimentary cDNA molecules probed for the desired miRNA markers.

Detection can be through any technique known in the field of molecular biology for detecting nucleic acids. Thus, it can be through agarose gel electrophoresis and staining with a nucleic acid specific stain. It can be through labeling of one or more of the primers with a detectable moiety, such as a fluorescent or radioactive molecule to produce a labeled product. Likewise, it can be through labeling with a member of a two-component label system, such as the digoxigenin system. Other non-limiting examples include detection based on column chromatography (e.g., size exclusion chromatography), mass spectrometry, and sequencing. Yet other non-limiting techniques include amplification of signal by enzymatic techniques and use of antibodies that are specific to a label attached to one or more nucleotides of the product to be detected. It can also be through real-time monitoring of luminescence/fluorescence as amplification proceeds. Those of skill in the art are well aware of the various techniques for detecting nucleic acids, and the various devices, supplies, and reagents that can be used to do so.

Detection can result in qualitative identification, semi-quantitative identification, or quantitative identification of the target miRNA. Qualitative detection includes detection of the presence or absence of a cDNA or amplification product, without any correlation to an amount of target miRNA in the sample that was tested. Qualitative results enable the practitioner to conclude that the target miRNA was present or absent in the sample, but do not enable him to ascertain the amount. Semi-quantitative detection permits not only detection of a signal, but correlation of the signal to a basal level of target miRNA in the sample that was tested. For example, it may indicate a minimum threshold amount of miRNA was present in the sample. Such a result enables the practitioner to determine if a pre-defined amount of miRNA target is present in the sample, but not to determine if less than that amount is present. Likewise, it does not enable the practitioner to determine the precise concentration or amount of miRNA in the original sample. Quantitative detection permits the practitioner to determine the amount of target miRNA present in the original sample over a wide range of amounts. In general, quantitative detection compares the amount detected to a reference or standard that is either previously generated (e.g., a standard curve) or generated at the time of the assay for the target miRNA using internal controls. Numerous techniques for performing quantitative and semi-quantitative analyses are known to those of skill in the art, and need not be detailed here. For example, those of skill in the art may consult various commercial products for suitable techniques for performing PCR, QPCR, generating standard curves, and quantitating and validating amplification results.

The method may comprise one or more additional optional steps as well. For example, nucleic acids or other substances can be purified to any extent prior to or at any time during the method, including as part of one or more steps, such as the detecting step. Likewise, inhibitors that might be present in one or more compositions can be removed by purification of the nucleic acids of the invention from the inhibitors. Such purification can be performed between two or more other steps of the method, as described herein (although it is to be understood that the steps described herein are not to be understood as limited in practice to the particular order in which they are presented or discussed). In addition, portions of one or more compositions formed during practice of the method may be removed. These can be used for any purpose, including, but not limited to, performing control reactions to ensure that one or more steps in the method are functioning properly, assaying for one or more substances in the composition to ensure that it is present, preferably in the amount expected, and determining any other reaction parameter of interest.

The method comprises amplification of the cDNA or reverse transcriptase product prior to, or at the time of, detection. Amplification of the cDNA can be performed using any suitable amplification technique, including, but not limited to, PCR and all of its variants (e.g., real-time PCR or quantitative PCR). In embodiments, the method comprises providing at least one amplification oligonucleotide primer, exposing the cDNA, if present, to the amplification primer, and exposing the resulting mixture to conditions that permit amplification of the single cDNA, if present. Preferably, oligonucleotides used for amplification comprise a miRNA-specific forward primer and a universal reverse primer. Therefore, in this case, the universal reverse primer comprises part of the sequence found in the reverse transcription primer (or adapter primer). In other embodiments, the adapter primer used for the reverse transcription reaction can also be employed in the amplification reaction.

The meaning of universal reverse primer in this context is any oligonucleotide sequence that can be used for PCR amplification of at least one miRNA and/or mRNA. The meaning of universal reverse primer is not meant to refer to any specific sequence but is meant to refer to a sequence that will be found in most if not all of the cDNA species produced by reverse transcription. Therefore, the sequence can be found universally in the population of cDNA species. Any specific sequence can be used in the universal reverse primer for PCR amplification.

### A. Identification of Mycobacterium

Provided herein are methods for detecting the presence of mycobacterial disease in a subject. The genus Mycobacterium includes pathogens known to cause serious diseases in mammals, including, for example, tuberculosis and leprosy. Mycobacterium (also referred to as mycobacteria) do not contain endospores or capsules, and are usually considered Gram-positive. In addition to the usual fatty acids found in membrane lipids, mycobacteria have a wide variety of very long-chain saturated (C₁₈-C₃₂) and monounsaturated (up to C₂₆) n-fatty acids. The occurrence of α-alkyl β-hydroxy very long-chain fatty acids, i.e., mycolic acids, is a hallmark of mycobacteria and related species. Mycobacterial mycolic acids are large (C₇₀-C₉₀) with a large α-branch (C₂₀-C₂₅). The main chain contains one or two double bonds, cyclopropane rings, epoxy groups, methoxy groups, keto groups or methyl branches. Such acids are major components of the cell wall, occurring mostly esterified in clusters of four on the terminal hexa-arabinofuranosyl units of the major cell-wall polysaccharides called arabinogalactans. They are also found esterified to the 6 and 6′ positions of trehalose to form ‘cord factor’. Small amounts of mycolate are also found esterified to glycerol or sugars such as trehalose, glucose and fructose depending on the sugars present in the culture medium. Mycobacteria also contain a wide variety of methyl-branched fatty acids. These include 10-methyl C₁₈ fatty acid (tuberculostearic acid found esterified in phosphatidyl inositide mannosides), 2,4-dimethyl C₁₄ acid and mono-, di- and trimethyl-branched C₁₄ to C₂₅ fatty acids found in trehalose-containing lipooligosaccharides, trimethyl unsaturated C₂₇ acid (phthienoic acid), tetra-methyl-branched C₂₈-C₃₂ fatty acids (mycocerosic acids) and shorter homologues found in phenolic glycolipids and phthiocerol esters, and multiple methyl-branched phthio-ceranic acids such as hepamethyl-branched C₃₇ acid and oxygenated multiple methyl-branched acids such as 17-hydroxy-2,4,6,8,10,12,14,16-octamethyl C₄₀ acid found in sulpholipids. In addition, mycocerosic acids and other branched acids are esterified to phthicerol and phenolphthicerol and their derivates. Kolattukudy et al., Mol. Microbio. 24(2):263-270 (1997). Evidence implicates specific cell envelope lipids in Mtb pathogenesis. Rao, et al., J. Exp. Med., 201(4):535-543 (2005).

Mycobacterium species include, but are not limited to: M. abscessus; M. africanum; M. agri; M. aichiense; M. alvei; M. arupense; M. asiaticum; M. aubagnense; M. aurum; M. austroafricanum; Mycobacterium avium complex (MAC); M. avium; M. avium paratuberculosis, which has been implicated in Crohn's disease in humans and Johne's disease in cattle and sheep; M. avium silvaticum; M. avium “hominissuis”; M. colombiense; M boeitickei; M. bohemicum; M. bolletii; M. botniense; M. bovis; M. branderi; M brisbanense; M. brumae; M. canariasense; M. caprae; M. celatum; M. chelonae; M chimaera; M. chitae; M. chlorophenolicum; M. chubuense; M. conceptionense; M confluentis; M. conspicuum; M. cookii; M. cosmeticum; M. diernhoferi; M. doricum; M duvalii; M. elephantis; M. fallax; M. farcinogenes; M. lavescens; M. lorentinum; M fluoroanthenivorans; M. fortuitum; M. fortuitum subsp. acetamidolyticum; M frederiksbergense; M. gadium; M. gastri; M. genavense; M. gilvum; M. goodii; M. gordonae; M haemophilum; M. hassiacum; M. heckeshornense; M. heidelbergense; M. hiberniae; M hodleri; M. holsaticum; M. houstonense; M. immunogenum; M. interjectum; M intermedium; M. intracellulare; M. kansasii; M. komossense; M. kubicae; M kumamotonense; M. lacus; M. lentiflavum; M. leprae, which causes leprosy; M lepraemurium; M. madagascariense; M. mageritense; M. malmoense; M. marinum; M massiliense; M. microti; M. monacense; M. montepiorense; M. moriokaense; M mucogenicum; M. murale; M. nebraskense; M. neoaurum; M. neworleansense; M nonchromogenicum; M. novocastrense; M. ohuense; M. palustre; M. parafortuitum; M parascrofulaceum; M. parmense; M. peregrinum; M. phlei; Mphocaicum; M. pinnipedii; M porcinum; M. poriferae; M. pseudoshottsii; M. pulveris; M. psychrotolerans; M. pyrenivorans; M. rhodesiae; M. saskatchewanense; M. scrofidaceum; M. senegalense; M seoulense; M. septicum; M. shimoidei; M. shottsii; M. simiae; M. smegmatis; M. sphagni; M szulgai; M. terrae; M. thermoresistibile; M. tokaiense; M. triplex; M. triviale; Mycobacterium tuberculosis complex (MTBC), members are causative agents of human and animal tuberculosis (M. tuberculosis, the major cause of human tuberculosis; M. bovis; M. bovis BCG; M. africanum; M. canetti; M. caprae; M. pinnipedii′); M. tusciae; M. ulcerans, which causes the “Buruli”, or “Bairnsdale, ulcer”; M. vaccae; M. vanbaalenii; M. wolinskyi; and M. xenopi.

Mycobacteria can be classified into several groups for purpose of diagnosis and treatment, for example: M. tuberculosis complex (MTB) which can cause tuberculosis: M. tuberculosis, M africanum, M bovis, M. bovis BCG, M caprae, M. microti, M pinnipedii, the dassie bacillus, and M. canettii (proposed name) (Somoskovi, et al., J Clinical Microbio 45(2):595-599 (2007)); M. leprae which causes Hansen's disease or leprosy; nontuberculous mycobacteria (NTM) are all the other mycobacteria which can cause pulmonary disease resembling tuberculosis, lymphadenitis, skin disease, or disseminated disease. MTB members show a high degree of genetic homogeneity. Somoskovi (2007). The mycobacteria of the invention is selected from a group consisting of mycobacterial disease, including but not restricted to Johne's disease, tuberculosis and Crohn's disease, or any other disease caused by Mycobacterium avium subspecies paratuberculosis (MAP) or any other species within the Mycobacterium genus.

### 1. Johne's Disease

Provided herein are methods of detecting mycobacterial disease, including Johne's disease. Mycobacterium paratuberculosis causes Johne's disease (paratuberculosis) in dairy cattle. The disease is characterized by chronic diarrhea, weight loss, and malnutrition, resulting in estimated losses of $220 million per year in the USA alone. World-wide, the prevalence of the disease can range from as low as 3-4% of the examined herds in regions with low incidence (such as England), to high levels of 50% of the herds in some areas within the USA (Wisconsin and Alabama). Cows infected with Johne's disease are known to secrete Mycobacterium paratuberculosis in their milk. In humans, M. paratuberculosis bacilli have been found in tissues examined from Crohn's disease patients indicating possible zoonotic transmission from infected dairy products to humans.

Unfortunately, the virulence mechanisms controlling M paratuberculosis persistence inside the host are poorly understood, and the key steps for establishing the presence of paratuberculosis are elusive. Mechanisms responsible for invasion and persistence of M. paratuberculosis inside the intestine remain undefined on a molecular level (Valentin-Weigand and Goethe, 1999, Microbes & Infection 1: 1121-1127). Both live and dead bacilli are observed in sub-epithelial macrophages after uptake. Once inside the macrophages, M paratuberculosis survive and proliferate inside the phagosomes using unknown mechanisms.

M. paratuberculosis is closely related to Mycobacterium avium subspecies avium (hereinafter referred to as Mycobacterium avium or M. avium), which is a persistent health problem for immunocompromised humans, particularly HIV-positive individuals. Limited tools are available to researchers to definitively identify M paratuberculosis and to distinguish it from M. avium. Existing methods are subject to high cross-reactivity, poor sensitivity, specificity, and predictive value. This dearth of knowledge translates into a lack of suitable vaccines for prevention and treatment of Johne's disease in animals, and of Crohn's disease in humans.

Many clinical methods for detecting and identifying Mycobacterium species in samples require analysis of the bacterium's physical characteristics (e.g., acid-fast staining and microscopic detection of bacilli), physiological characteristics (e.g., growth on defined media) or biochemical characteristics (e.g., membrane lipid composition). These methods require relatively high concentrations of bacteria in the sample to be detected, may be subjective depending on the clinical technician's experience and expertise, and are time-consuming. Because Mycobacterium species are often difficult to grow in vitro and may take weeks to reach a useful density in culture, these methods can also result in delayed patient treatment and costs associated with isolating an infected individual until the diagnosis is completed.

More recently, assays that detect the presence of nucleic acid derived from bacteria in the sample have been preferred because of the sensitivity and relative speed of the assays. In particular, assays that use in vitro nucleic acid amplification of nucleic acids present in a clinical sample can provide increased sensitivity and specificity of detection. Such assays, however, can be limited to detecting one or a few Mycobacterium species depending on the sequences amplified and/or detected.

### B. Identification of Target Sequences

The present invention relates to a method for detecting the presence or amount of a target polynucleotide (nucleic acid sequence) from Mycobacterium paratuberculosis in a sample. The target polynucleotide is a virulence determinant. In a preferred embodiment, the target polynucleotide is a miRNA. The invention is also directed to a method of detecting the presence of a disease state in a mammal, by detecting the presence or amount of a target miRNA, wherein the presence or amount of the target miRNA identifies the disease state. Thus, the invention relates to diagnostic compositions and methods for detecting Johne's disease. The sample containing the target miRNA may be tissue, collection of cells, cell lysate, body fluid, excretum, in vitro culture, purified polynucleotide, isolated polynucleotide, food sample, medical sample, agro-livestock sample, or environmental sample.

The invention described here utilizes large-scale identification of disrupted genes and the use of bioinformatics and AI to select mutants that could be characterized in animals.

Identification of target sequences of the present invention may be accomplished by a number of techniques. For instance, oligonucleotide probes based on the sequences disclosed here can be used to identify the desired gene in a cDNA or genomic DNA library from a desired bacterial strain. To construct genomic libraries, large segments of genomic DNA are generated by random fragmentation, e.g. using restriction endonucleases, and are ligated with vector DNA to form concatemers that can be packaged into the appropriate vector. The cDNA or genomic library can then be screened using a probe based upon the sequence of a cloned gene such as the polynucleotides disclosed here. Probes may be used to hybridize with genomic DNA or cDNA sequences to identify homologous genes in the same or different bacterial strains.

Alternatively, the nucleic acids of interest can be amplified from nucleic acid samples using amplification techniques. For instance, polymerase chain reaction (PCR) technology can be used to amplify the sequences of the genes directly from mRNA, from cDNA, from genomic libraries or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes.

Appropriate nucleic acids for identifying the target sequences of the present invention from a sample are generated from comparisons of the sequences provided herein, according to standard PCR guides. For examples of the miRNAs used see Table 1 and Table 2.

Polynucleotides may also be synthesized by well-known techniques described in the technical literature. Double-stranded DNA fragments may then be obtained either by synthesizing the complementary strand and annealing the strands together under appropriate conditions, or by adding the complementary strand using DNA polymerase with an appropriate primer sequence.

Once a nucleic acid is isolated using the method described above, standard methods can be used to determine if the nucleic acid is a preferred nucleic acid of the present invention, e.g., by using structural and functional assays known in the art. For example, using standard methods, the skilled practitioner can compare the sequence of a putative nucleic acid sequence thought to encode a preferred protein of the present invention to a nucleic acid sequence encoding a preferred protein of the present invention to determine if the putative nucleic acid is a preferred polynucleotide of the present invention.

Gene amplification and/or expression can be measured in a sample directly, for example, by conventional Southern blotting, Northern blotting to quantitate the transcription of mRNA, dot blotting (DNA analysis), DNA microarrays, or in situ hybridization, using an appropriately labeled probe, based on the sequences provided herein. Various labels can be employed, most commonly fluorochromes and radioisotopes, particularly ³²P. However, other techniques can also be employed, such as using biotin-modified nucleotides for introduction into a polynucleotide. The biotin then serves as the site for binding to avidin or antibodies, which can be labeled with a variety of labels, such as radionuclides, fluorescers, enzymes, or the like. Alternatively, antibodies can be employed that can recognize specific duplexes, including DNA duplexes, RNA duplexes, DNA-RNA hybrid duplexes or DNA-protein duplexes. The antibodies in turn can be labeled and the assay can be carried out where the duplex is bound to a surface, so that upon the formation of duplex on the surface, the presence of antibody bound to the duplex can be detected.

Gene expression can also be measured by immunological methods, such as immunohistochemical staining. With immunohistochemical staining techniques, a sample is prepared, typically by dehydration and fixation, followed by reaction with labeled antibodies specific for the gene product coupled, where the labels are usually visually detectable, such as enzymatic labels, fluorescent labels, luminescent labels, and the like. Gene expression can also be measured using PCR techniques, or using DNA microarrays, commonly known as gene chips.

### C. Preparation of Total RNA

The methods presented herein describe the detection of miRNA by proxy of generated cDNA. Compared to the direct detection of miRNA within an extracted sample, the additional step of cDNA synthesis provides an amplification stage, increasing sensitivity of detection in low volume or low expression samples.

The disclosed methods can be used to prepare cDNA libraries that are representative the total RNA, mRNA or miRNA present in the cell or cells from which the RNA starting material is prepared. Typically, miRNA starting materials is accessed by lysing or otherwise disrupting one or more cells of interest under conditions that prevent loss or degradation of miRNA. For example, the conditions or buffers used can include reagents (e.g., inhibitors) be carried out under conditions that reduce or inhibit the activity of RNase.

Preferably the RNA starting material is separated from genomic DNA. In some embodiments, the RNA is isolated from the cell lysate for uses in the subsequent steps of the cDNA library preparation. In some embodiments, the genomic DNA is removed from the cell lysate, and the cell lysate, including total cellular mRNA is utilized as the starting material for reverse transcription. In some embodiments, isolation of total RNA and removal of genomic DNA are combined.

Methods and kits for facilitating RNA isolation, and/or removal of genomic DNA are known in the art and can be used or modified as discussed herein to facilitate preparation of RNA for reverse transcription. In the methods disclosed herein, when used as per the manufacturer's recommendation below, you have direct capture/detection of miRNA without amplification, and with the potential multiplexing up to 80 (or 150 depending on the Luminex machine used) individual markers at once.

The process typically includes spinning cell or tissue lysates through spin columns to remove genomic DNA. Next, total RNA is purified using a second spin column. In a preferred embodiment, an RNA carrier, such synthetic poly(A) RNA, can added to the lysis buffer before homogenizing the cells.

The cells are lysed under mild conditions that breakdown the cell's plasma membrane, but leave the nuclear membrane substantially or completely intact. In this way starting, cytoplasmic RNA can be more easily and completely harvested from the genomic DNA that can contaminate or otherwise corrupt preparation of cDNA library. In this way the column purification is of RNA is prevented and consequently the RNA included with the cytoplasm can be more efficiently recovered. The RNA harvested in this are enriched for cytoplasmic RNA relative to nuclear RNA. The methods can include a centrifugation sequence wherein the supernatant containing cytoplasmic RNA is recovered for RT.

Mild lysis buffer can include one or more detergents such as TRIXTON®-X100, IGEPAL CA-630, NP40, TWEEN® 20 at a concentration of about 0.01 to about 2%. Buffer can include TCEP (tris(2-carboxyethyl)phosphine). The lysis can be carried out are room temperature, or for a shorter period of time at warmer temperature. The lysis is typically carried out for between about 10 minutes and 2 hours at a temperature between about 4° C. and 75° C. A DNA endonuclease can be applied to digest the genomic DNA, and the RNA retained and used for RT.

The reverse transcription and optionally cDNA amplification can be carried out without removing genomic DNA or without purifying RNA (and/or cDNA). For example, in some embodiments, a “single tube” protocol is employed wherein cell lysis, reverse transcription, optionally cDNA amplification, and optionally subsequent steps are carrier out in a single tube. Therefore, one or more of RT and optional second strand synthesis, end-blunting, phosphorylation and ligation, the amplification (e.g., using phi29 DNA polymerase and its related components) can be performed in the same tube. In a particular embodiment, during each step the reaction volume is enlarged (usually doubled) and the buffer is adjusted, without purification until the cDNA amplicon is obtained and the cDNA amplification is completed. In some embodiments, the enzyme(s) are inactivated between one or more of the steps. In some embodiments, none of the enzymes are inactivated.

It is generally understood that as more strict RNA purification is employed there will be less contamination by genomic DNA, but more of the total RNA will also be lost. This can lead to a reduction in low frequency transcripts below what can be suitably amplified as cDNA. Accordingly, in some embodiments, few or no purification steps are employed with the RNA starting material is being prepared from between 1 and 10,000 cells, between 1 and 1,000 cells, between 1 and 500 cells, between 1 and 100 cells, between 1 and 50 cells, between 1 and 10 cells, or 1 cell. Alternatively, in some embodiments, one or more steps of RNA purification or genomic DNA removal are employed when the RNA starting material is being prepared from 1 cell, more preferably between 1 and 10 cells, more preferably between 1 and 50 cells, more preferably between 1 and 100 cells, more preferably between 1 and 500 cells, more preferably greater than 1,000 cells, more preferably greater than 10,000 cells, most preferably greater than 10,000 cells.

D. Multiplex miRNA Profiling

In one embodiment, the method uses FirePlex® particles, which enable the multiplex capture of miRNAs with picomolar sensitivity and high specificity. The FirePlex® particles contain three distinct functional regions that are separated from each other by inert spacer regions. The central region of each particle is known as a central analyte or miRNA quantification region which contains miRNA probes that can capture target miRNAs. The central region of the particle comprises a reporter dye. The two end regions of each particle act as two halves of a barcode that distinguish between different particles. Detection is carried out using a flow cytometer to detect miRNA molecules that emit fluorescence that is proportional to their abundance in the sample. The flow cytometer was used to detect the fluorescence signal from the center of each particle through the reporter dye. Each miRNA that was used was given a unique code (up to 70 different codes were possible). The data that was obtained from the mixture of particles could then be attributed to the miRNAs by identification of the code.

The FirePlex® miRNA Assay enables high-throughput detection of up to 65 miRNA targets by flow cytometry, and streamlined analysis with the FirePlex® Analysis Workbench Software. The assay is performed in 96-well plate format such that users can detect up to 65 miRNA targets for each of 96 samples in less than a day's work. The FirePlex® miRNA Assay provides PCR sensitivity while eliminating the need for separate reverse transcription reactions and mitigating amplification biases introduced by target-specific PCR. This is made possible by combining uniquely encoded hydrogel particles with single-step RT-PCR amplification using universal primers. The assay reliably detects as few as 1000 miRNA copies per sample with a linear dynamic range of −5 logs. In addition to increased sensitivity, multiplexed detection using the FirePlex® platform conserves precious sample by detecting multiple miRNA targets from as little as: 100 pg of purified RNA; or directly from 10 μL biofluids including serum and plasma, without RNA isolation; or from 5 micron sections of tissues and FFPE samples (Tackett M R, Diwan I., Methods Mol Biol. 2017; 1654:209-219.)

E. Luminex xMAP System with xTAG Beads

The disclosed method provides direct capture/detection of miRNA without amplification, and with the potential multiplexing up to 80 (or 150 depending on the Luminex machine used) individual markers at once. Samples are prepared using an RNA extraction kit such as Qiagen miRNeasy Serum/Plasma Advanced Kit. Serum (and other body fluids) often have only low concentrations of miRNA, and initial studies suggest that a minimum of 600 ul of serum (therefore at least twice as much whole blood may be required to extract the minimum total RNA concentration for the system), this is impractical for the sampling of small animals such as cats, etc. Therefore, there remains a need to provide a solution to the low serum volume/low RNA concentration issue.

1. MicroRNA Analysis with Luminex xMAP Outline (from Luminex Handbook)

A number of PCR-based and direct hybridization assays are available for the analysis of miRNA expression levels. Most of the PCR-based approaches can only be run as singleplex assays in individual reactions or on costly chips, increasing processing times, requiring more sample, and limiting the number of samples that can be processed rapidly.

Hybridization assays can be multiplexed to different degrees with the use of special costly probes, cassettes, and analysis instruments. Many of these chemistries are suitable for analysis of expression levels, but often lack the ability to distinguish between closely related miRNA targets that differ by a single base. In addition to the lack of single base resolution, these assays can also be costly per sample, with low sample throughput capabilities.

To overcome these obstacles, the Luminex-based nuclease protection approach takes advantage of a unique combination of three essential assay characteristics:
1. Use of MagPlex-TAG™ Microspheres. Users can create their own mixes as needed. These magnetic beads are available from Luminex with unique TAG sequences already coupled to them. These sequences are universal array sequences that do not cross-hybridize with each other or with native sequences.
2. Biotin-labeled chimeric probes. These are composed of RNA sequences that are 100% complementary to their mature miRNA targets, and a DNA sequence which is 100% complementary to specific anti-TAG sequences on the MagPlex-TAG Microspheres. The probes can be easily designed by the user, making the assay more cost effective and flexible.
3. Nuclease protection chemistry. This chemistry, when combined with the assay's step-down hybridization protocol, results in single-base resolution of nucleotide differences, even in miRNA species that cannot be distinguished with other chemistries. This combination of characteristics also contributes to the miRNA assay having a short one-day protocol without sacrificing single nucleotide specificity, even without PCR amplification. This is achieved by the ability of the biotinylated chimeric probes to specifically bind their miRNA targets in a short period of time with the protocol's step-down hybridization approach. The chimeric probe/miRNA complexes are then rapidly captured on MagPlex-TAG beads, followed by a short nuclease reaction that degrades mismatched and unbound probes. Following a short SAPE labeling step and some washes, the samples are ready for analysis. The methods disclosed herein are based on the former Luminex FLEXMIR® v2 product and use SAPE as the reporter. The optimal reporter concentration should be determined by titration.
   F. Detection by Proxy—Translate miRNA to cDNA then Detect cDNA Expression Levels

The methods disclosed herein provide a solution to the low serum volume/low RNA concentration issue discussed above. There are a number of commercial kits such as TaqMan™ Advanced miRNA cDNA Synthesis Kit, that can be used to translate a RNA isolation preparation (with serum this could be a simple as a Proteinase K incubation) in to cDNA (so the cDNA for all RNA present in the sample). This cDNA is then amplified by PCR to a detectable concentration.

This cDNA preparation can then be probed for the cDNA complimentary to the desired miRNA markers, therefore inferring the expression levels of particular miRNA through the detection of its cDNA; detection by proxy.

The workflow in the methods disclosed herein is as follows: RNA extraction of biofluid>translation of RNA to cDNA with PCR amplification of cDNA>detection of cDNA in amplified sample.

The detection of a specific cDNA could be done using Luminex xMAP for a large multiplexing capacity, or by simpler methods such as microarrays or RT-PCR for small multiplex or singleplex reactions. Detection of cDNA can be done using RNA or DNA probes. The Luminex xMAP protocol is adapted for cDNA detection by substitution of a DNAse for the RNAase recommended in the Luminex xMAP protocol. Examples of a DNAase that may be used include, but are not limited to, S1 nuclease, P1 nuclease or any other DNAase which will digest single strand DNA.

Commonplace methods for miRNA detection use a version this ‘detection by proxy’ method routinely, e.g. Next Generation Sequencing, RT-PCR. The efficiencies of conversion of RNA to cDNA may not be consistent across all RNAs, i.e. a particular cDNA may be expressed at a high or lower level than the original RNA. For the purposes of methods provided herein this is not necessarily an issue—the method identifies relative levels of expression of RNA between each other, and correlates this pattern to clinical status. The absolute miRNA express values are not required as long as any expression efficiencies are consistent.

### G. Predictive Modelling

Provided herein are methods using predictive modelling to investigate the scope to use the miRNA profiles to predict the presence or absence of disease. A group of healthy and unhealthy animals were taken and tested to determine the level of miRNA cDNA expression in samples from these animals. The data obtained was then used to train the models.

Fifteen machine learning models were fitted and compared with the aim of obtaining the best predictions of the disease outcome. An important consideration in respect of the data set for this example was the relatively large difference between the number of samples belonging to the different disease outcomes. In this case, a sampling procedure called SMOTE was used with the aim to correct for this unbalanced class problem while comparing the performance of the models. A number of statistics based on 5-time repeated 10-fold cross-validation were calculated for each model. Cross-validation was useful to obtain more realistic model performance measures from the training data.

Data from the FirePlex® analysis from each of the miRNA molecules presented herein was fitted to each of the models.

### H. Kits

Also provided herein is a kit for use in performing the method of the first aspect comprising means for determining the level of expression of each one of the following miRNA molecules by proxy of the corresponding cDNA: bta.mir.433, bta.mir.146a, and bta.mir.29b.

There is also provided a method of selecting a panel for use in disease diagnosis comprising the steps of: (a) selecting a group of miRNA molecules the differential expression of which may be associated with a disease condition; (b) transcribing the miRNA into cDNA; (c) determining the levels miRNA based on the cDNA in the sample; and (d) training one or more AI model to be able to predict the disease condition.

### EXAMPLES

### Example 1: miRNA Extraction from Biological Sample

### Materials and Methods

This protocol describes the detection of miRNA by proxy of generated cDNA. Compared to the direct detection of miRNA within an extracted sample, the additional step of cDNA synthesis provides an amplification stage, increasing sensitivity of detection in low volume or low expression samples. In one embodiment miRNA molecules were selected from a group nucleotide sequences having least 90%, 95%, 97%, 98% or 99% or less to SEQ ID NO: 1-24 (Table 1). In another embodiment the normalizer miRNA molecules were selected from a group nucleotide sequences having least 90%, 95%, 97%, 98% or 99% or less to SEQ ID NO:25, 26, 27, 28 and 29. In one other embodiment the off species control miRNA molecules were selected from a group nucleotide sequences having least 90%, 95%, 97%, 98% or 99% or less to SEQ ID NO:30, 31, and 32.

TABLE 1
miRNA sequence
Probes

| | | SEQ ID |
|---|---|---|
| miRNA Name | miRNA sequence | NO: |
| bta.mir.146a | UGAGAACUGAAUUCCAUAGGUUGU | 1 |
| bta.mir.433 | AUCAUGAUGGGCUCCUCGGUGU | 2 |
| bta.mir.29b | UAGCACCAUUUGAAAUCAGUGUU | 3 |
| bta.mir.105a | UCAAAUGCUCAGACUCCUGUGGU | 4 |
| bta.mir.100 | AACCCGUAGAUCCGAACUUGUG | 5 |
| bta.mir.1247.5p | ACCCGUCCCGUGCGUCCCCGGA | 6 |
| bta.mir.155 | UUAAUGCUAAUCGUGAUAGGGGU | 7 |
| bta.mir.6517 | UCAGGGUCCGUGAGCUCCUCGGC | 8 |
| bta.mir.24 | GUGCCUACUGAGCUGAUAUCAGU | 9 |
| bta.mir.184 | UGGACGGAGAACUGAUAAGGGU | 10 |
| bta.mir.142.3p | AGUGUUUCCUACUUUAUGGAUG | 11 |
| bta.mir.137 | UUAUUGCUUAAGAAUACGCGUAG | 12 |
| bta.mir.582 | UUACAGUUGUUCAACCAGUUACU | 13 |
| bta.mir.196b | UAGGUAGUUUCCUGUUGUUGGGA | 14 |
| bta.mir.19b | UGUGCAAAUCCAUGCAAAACUGA | 15 |
| bta.mir.21.5p | UAGCUUAUCAGACUGAUGUUGACU | 16 |
| bta.mir.133b | UUUGGUCCCCUUCAACCAGCUA | 17 |
| bta.mir.378c | ACUGGACUUGGAGUCAGAAGU | 18 |
| bta.mir.32 | UAUUGCACAUGACUAAGUUGCAU | 19 |
| bta.mir.202 | UUCCUAUGCAUAUACUUCUUU | 20 |
| bta.mir.1271 | CUUGGCACCUAGUAAGUACUCA | 21 |
| bta.mir.7857.5p | AUAGCCAGUUGGGGAAGAAUGC | 22 |
| bta.mir.29a | CUAGCACCAUCUGAAAUCGGUUA | 23 |
| bta.miR.301a | CAGUGCAAUAGUAUUGUCAAAGCAU | 24 |

Normalizers

| | | SEQ ID |
|---|---|---|
| miRNA Name | miRNA sequence | NO: |
| bta.mir.93 | CAAAGUGCUGUUCGUGCAGGUA | 25 |
| bta.mir.20a.5p | UAAAGUGCUUAUAGUGCAGGUAG | 26 |
| bta.mir.16a.5p | UAGCAGCACGUAAAUAUUGGUG | 27 |
| bta.mir.17.5p | CAAAGUGCUUACAGUGCAGGUAGU | 28 |
| bta.mir.92a | UAUUGCACUUGUCCCGGCCUGU | 29 |

Off-species controls

| | | SEQ ID |
|---|---|---|
| miRNA Name | miRNA sequence | NO: |
| cel.mir.70.3p | UAAUACGUCGUUGGUGUUUCCAU | 30 |
| oan.mir.7417.5p | UUCCCCACUCUGAGCACACAGC | 31 |
| ath.mir167d | UGAAGCUGCCAGCAUGAUCUGG | 32 |

In this experiment, 5 probes were used comprised as probes for bta.mir.433, bta.mir.146a, bta.mir.29b, with two normalizers of bta.mir.92a and bta.mir.20a.5p

A total of 15 Johne's positive serum samples, 14 Johne's negative serum samples, one serum sample of unknown status and three non-template controls (NTC) were run in triplicate.

Stage 1—miRNA extraction from biological sample: MircoRNAs were extracted from the provided biological sample (blood, serum, plasma, urine, milk, etc.) using commercially available miRNA specific extraction kits and the manufacturer's recommended protocol, e.g. Qiagen miRNeasy Serum/Plasma Kit using the protocol in the manufacturer's handbook.

Stage 2—cDNA synthesis: From the extracted miRNA, cDNA and the reverse compliment cDNA are synthesized and amplified using commercially available miRNA specific extraction kits and the manufacturer's recommended protocol, e.g. TaqMan Advanced miRNA cDNA Synthesis Kit using the protocol in the manufacturer's handbook. In one embodiment cDNA molecules were selected from a group nucleotide sequences having least 90%, 95%, 97%, 98% or 99% or less to SEQ ID NO: 33-56 (Table 2). In another embodiment the normalizer cDNA molecules were selected from a group nucleotide sequences having least 90%, 95%, 97%, 98% or 99% or less to SEQ ID NO:57, 58, 59, 60 and 67. In one other embodiment the off species control cDNA molecules were selected from a group nucleotide sequences having least 90%, 95%, 97%, 98% or 99% or less to SEQ ID NO:62, 63 and 64.

TABLE 2
cDNA and Reverse Compliment cDNA sequences.
Probes

| | | SEQ ID | Reverse compliment | SEQ ID |
|---|---|---|---|---|
| miRNA Name | cDNA sequence | NO: | cDNA sequence | NO: |
| bta.mir.146a | TGAGAACTGAAT | 33 | ACAACCTATGGAAT | 65 |
| | TCCATAGGTTGT | | TCAGTTCTCA | |
| bta.mir.433 | ATCATGATGGGC | 34 | ACACCGAGGAGCCC | 66 |
| | TCCTCGGTGT | | ATCATGAT | |
| bta.mir.29b | TAGCACCATTTG | 35 | AACACTGATTTCAA | 67 |
| | AAATCAGTGTT | | ATGGTGCTA | |
| bta.mir.105a | TCAAATGCTCAG | 36 | ACCACAGGAGTCTG | 68 |
| | ACTCCTGTGGT | | AGCATTTGA | |
| bta.mir.100 | AACCCGTAGATC | 37 | CACAAGTTCGGATC | 69 |
| | CGAACTTGTG | | TACGGGTT | |
| bta.mir.1247.5p | ACCCGTCCCGTG | 38 | TCCGGGGACGCACG | 70 |
| | CGTCCCCGGA | | GGACGGGT | |
| bta.mir.155 | TTAATGCTAATC | 39 | ACCCCTATCACGATT | 71 |
| | GTGATAGGGGT | | AGCATTAA | |
| bta.mir.6517 | TCAGGGTCCGTG | 40 | GCCGAGGAGCTCAC | 72 |
| | AGCTCCTCGGC | | GGACCCTGA | |
| bta.mir.24 | GTGCCTACTGAG | 41 | ACTGATATCAGCTC | 73 |
| | CTGATATCAGT | | AGTAGGCAC | |
| bta.mir.184 | TGGACGGAGAAC | 42 | ACCCTTATCAGTTCT | 74 |
| | TGATAAGGGT | | CCGTCCA | |
| bta.mir.142.3p | AGTGTTTCCTACT | 43 | CATCCATAAAGTAG | 75 |
| | TTATGGATG | | GAAACACT | |
| bta.mir.137 | TTATTGCTTAAG | 44 | CTACGCGTATTCTTA | 76 |
| | AATACGCGTAG | | AGCAATAA | |
| bta.mir.582 | TTACAGTTGTTCA | 45 | AGTAACTGGTTGAA | 77 |
| | ACCAGTTACT | | CAACTGTAA | |
| bta.mir.196b | TAGGTAGTTTCCT | 46 | TCCCAACAACAGGA | 78 |
| | GTTGTTGGGA | | AACTACCTA | |
| bta.mir.19b | TGTGCAAATCCA | 47 | TCAGTTTTGCATGGA | 79 |
| | TGCAAAACTGA | | TTTGCACA | |
| bta.mir.21.5p | TAGCTTATCAGA | 48 | AGTCAACATCAGTC | 80 |
| | CTGATGTTGACT | | TGATAAGCTA | |
| bta.mir.133b | TTTGGTCCCCTTC | 49 | TAGCTGGTTGAAGG | 81 |
| | AACCAGCTA | | GGACCAAA | |
| bta.mir.378c | ACTGGACTTGGA | 50 | ACTTCTGACTCCAA | 82 |
| | GTCAGAAGT | | GTCCAGT | |
| bta.mir.32 | TATTGCACATGA | 51 | ATGCAACTTAGTCA | 83 |
| | CTAAGTTGCAT | | TGTGCAATA | |
| bta.mir.202 | TTCCTATGCATAT | 52 | AAAGAAGTATATGC | 84 |
| | ACTTCTTT | | ATAGGAA | |
| bta.mir.1271 | CTTGGCACCTAG | 53 | TGAGTACTTACTAG | 85 |
| | TAAGTACTCA | | GTGCCAAG | |
| bta.mir.7857.5p | ATAGCCAGTTGG | 54 | GCATTCTTCCCCAAC | 86 |
| | GGAAGAATGC | | TGGCTAT | |
| bta.mir.29a | CTAGCACCATCT | 55 | TAACCGATTTCAGA | 87 |
| | GAAATCGGTTA | | TGGTGCTAG | |
| bta.miR.301a | CAGTGCAATAGT | 56 | ATGCTTTGACAATA | 88 |
| | ATTGTCAAAGCA | | CTATTGCACTG | |
| | T | | | |

Normalizers

| | | SEQ ID | Reverse compliment | SEQ ID |
|---|---|---|---|---|
| miRNA Name | cDNA sequence | NO: | cDNA sequence | NO: |
| bta.mir.93 | CAAAGTGCTGTT | 57 | TACCTGCACGAACA | 89 |
| | CGTGCAGGTA | | GCACTTTG | |
| bta.mir.20a.5p | TAAAGTGCTTAT | 58 | CTACCTGCACTATA | 90 |
| | AGTGCAGGTAG | | AGCACTTTA | |
| bta.mir.16a.5p | TAGCAGCACGTA | 59 | CACCAATATTTACGT | 91 |
| | AATATTGGTG | | GCTGCTA | |
| bta.mir.17.5p | CAAAGTGCTTAC | 60 | ACTACCTGCACTGT | 92 |
| | AGTGCAGGTAGT | | AAGCACTTTG | |
| bta.mir.92a | TATTGCACTTGTC | 61 | ACAGGCCGGGACAA | 93 |
| | CCGGCCTGT | | GTGCAATA | |

Off-species controls

| | | SEQ ID | Reverse compliment | SEQ ID |
|---|---|---|---|---|
| miRNA Name | cDNA sequence | NO: | cDNA sequence | NO: |
| cel.mir.70.3p | TAATACGTCGTT | 62 | ATGGAAACACCAAC | 94 |
| | GGTGTTTCCAT | | GACGTATTA | |
| oan.mir.7417.5p | TTCCCCACTCTGA | 63 | GCTGTGTGCTCAGA | 95 |
| | GCACACAGC | | GTGGGGAA | |
| ath.mir167d | TGAAGCTGCCAG | 64 | CCAGATCATGCTGG | 96 |
| | CATGATCTGG | | CAGCTTCA | |

Stage 3—Expression profiling: The relative expression levels of specific miRNA, which form part of the defined diagnostic panel, are inferred through the relative expression levels of their respective cDNA, i.e. detection by proxy. This can be performed via numerous traditional DNA detection methods, such as PCR or Next Generation sequencing, or via newer multiplexing techniques such as beads capture technologies such as the Luminex xMAP system as using the protocol in the manufacturer's handbook. In this procedure the recommended RNAase was substituted with S1 nuclease (Thermo Fisher Scientific).

In another embodiment, the relative expression levels of specific miRNA, are inferred through the relative expression levels of their respective reverse compliment cDNA. In one embodiment reverse compliment cDNA molecules were selected from a group nucleotide sequences having least 90%, 95%, 97%, 98% or 99% or less to SEQ ID NO: 65-88 (Table 2). In another embodiment the normalizer reverse compliment cDNA molecules were selected from a group nucleotide sequences having least 90%, 95%, 97%, 98% or 99% or less to SEQ ID NO:89, 90, 91, 92 and 93. In one other embodiment the off species control cDNA molecules were selected from a group nucleotide sequences having least 90%, 95%, 97%, 98% or 99% or less to SEQ ID NO:94, 95, and 96.

Stage 4 Data analysis: The mean fluorescent intensities (MFI) of the results were initially normalized using the two normalizing probes as reference, and the lowest value MFI was limited to the NTC. Further refinement was then archived by omitting samples with an MFI lower than the NTC.

### Results

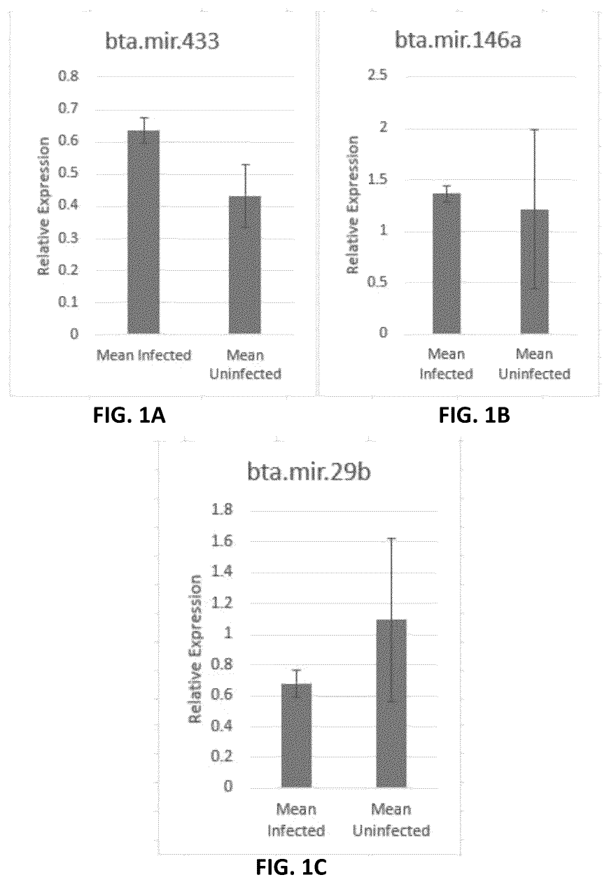

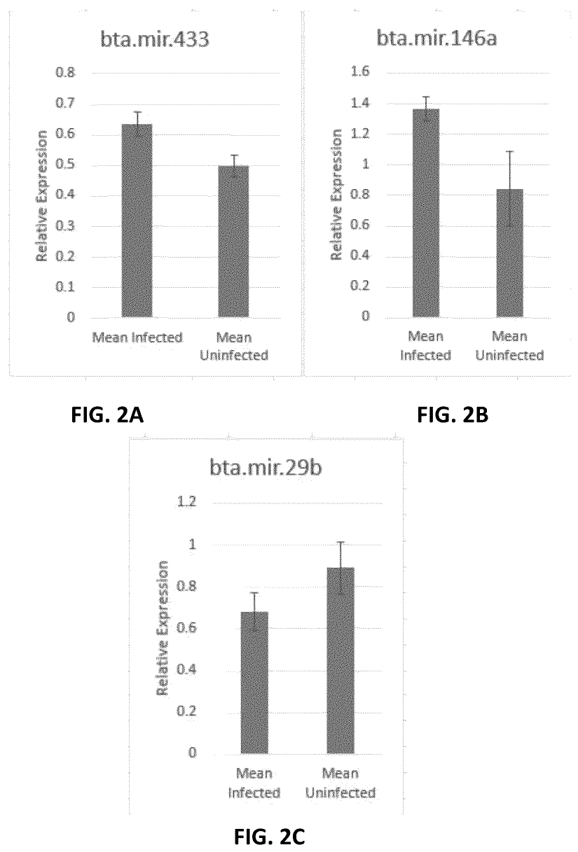

Initial analysis for the three probes, bta.mir.433, bta.mir.146a, and bta.mir.29b, resulted in a relative expression which was statistically significant and matched the expect direction for each group (FIGS. 1A-IC). To improve the error bars, the lowest value MFI for each miRNA probe was limited to the NTC removing a small number of the negative samples (FIGS. 2A-2C).

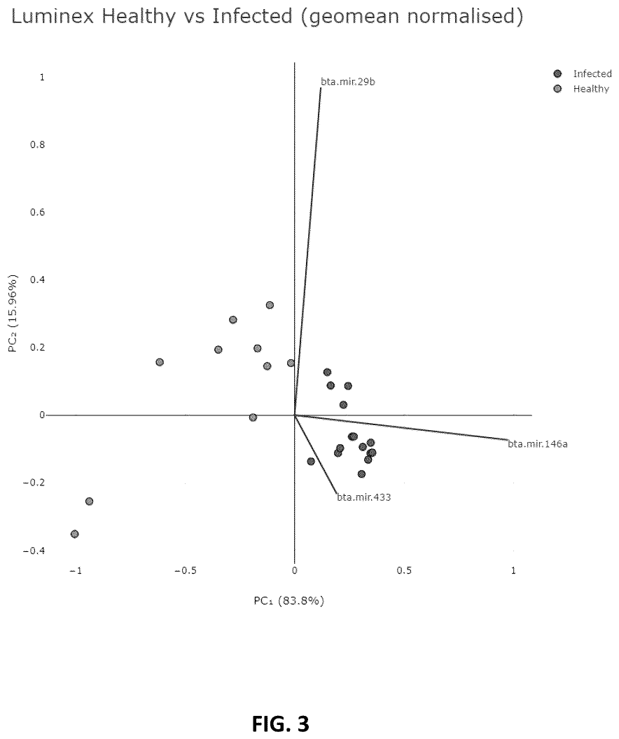

A PCA plot was generate to show sample groupings based on all three probes (FIG. 3). The unknown sample sat within the positive Johne's sample group. The modified cDNA detection protocol was able to successfully replicate expected results based on a low number of probes and samples of known status.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method for detecting the presence of mycobacterial disease in a subject, comprising the steps of:
(a) isolating miRNA molecules within a sample from a subject;
(b) translating the miRNA molecules to cDNA or molecules;
(c) amplifying the cDNA molecules to a detectable concentration;
(d) probing for the cDNA molecules complimentary to the desired miRNA markers;
(e) determining a level of expression of the miRNA molecules within a sample from a subject by the level of cDNA molecules probed for the desired miRNA markers; and
(f) using one or more Artificial Intelligence (AI) model to predict the disease condition of the subject.

2. The method according to claim 1, wherein the one or more AI model compares the level of expression of each cDNA molecule with at least one pre-determined reference level cDNA molecule characteristic of a non-diseased subject wherein a deviation of the level of expression of said cDNA molecule in comparison with the at least one reference level cDNA molecule allows for the diagnosis and/or prognosis of the disease.

3. The method according to claim 1, wherein the cDNA molecule may also be a reverse compliment cDNA.

4. The method according to claim 1, wherein the miRNA molecules are selected from a group consisting of nucleic acid sequence having at least 95%, 97%, 98% or 99% sequence identity to SEQ ID NO: 1, 2, 3, 4, 5, 6, 7, 8, 9, 10, 11, 12, 13, 14, 15, 16, 17, 18, 19, 20, 21, 22, 23 and 24.

5. The method of claim 1, wherein the cDNA molecules are selected from a group consisting of nucleic acid sequence having at least 95%, 97%, 98% or 99% sequence identity to SEQ ID NO:33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, and 88.

6. The method according to claim 1, wherein the method further comprises the use of at least one normalizer and/or control miRNA or cDNA molecule.

7. The method according to claim 6, wherein the at least one normalizer miRNA molecule is selected from a group consisting of nucleic acid sequence having at least 95%, 97%, 98% or 99% sequence identity to SEQ ID NO:25, 26, 27, 28 and 29.

8. The method according to claim 6, wherein the at least one normalizer cDNA molecule is selected from a group consisting of nucleic acid sequence having at least 95%, 97%, 98% or 99% sequence identity to SEQ ID NO:57, 58, 59, 60, 61, 89, 90, 91, 92, and 93.

9. The method according to claim 6, wherein the at least one of the control miRNA molecules is selected from a group consisting of nucleic acid sequence having at least 95%, 97%, 98% or 99% sequence identity to SEQ ID NO:30, 31, and 32.

10. The method according to claim 6, wherein the at least one of the control cDNA molecules is selected from a group consisting of nucleic acid sequence having at least 95%, 97%, 98% or 99% sequence identity to SEQ ID NO:62, 63, 64, 94, 95 and 96.

11. The method according to claim 1, wherein the method further comprises the step of using a machine learning algorithm for predictive modelling.

12. The method according to claim 1, wherein the method comprises the use of a combination of AI models.

13. The method according to claim 1, wherein the subject is a mammal.

14. The method according to claim 13, wherein the subject is a cow, sheep, goat, deer, llama, alpaca or vicuna.

15. The method according to claim 1, wherein the disease is selected from a group consisting of mycobacterial disease, including but not restricted to Johne's disease, tuberculosis and Crohn's disease, or any other disease caused by Mycobacterium avium subspecies paratuberculosis (MAP) or any other species within the Mycobacterium genus.

16. The method according to claim 1, wherein the sample is a biofluid selected from the group consisting of blood, urine, milk, tissue fluid, saliva, milk, cerebrospinal fluid (CSF), faeces or another biofluid.

17. The method according to claim 1, wherein the miRNA molecules are cell free miRNA molecules.

18. A method of selecting a panel for use in disease diagnosis comprising the steps of:
(a) selecting a group of miRNA molecules the differential expression of which may be associated with a disease condition;
(b) transcribing the miRNA into cDNA;
(c) determining the levels miRNA based on the cDNA in the sample; and
(d) training one or more AI model to be able to predict the disease condition.

19. A kit for use in performing the method of the claim 1 comprising means for determining the level of expression of each one of the following miRNA molecules by proxy of the corresponding cDNA: nucleic acid sequence having at least 95%, 97%, 98% or 99% sequence identity to SEQ ID NO: 1, 2, 3, 4, 5, 6, 7, 8, 9, 10, 11, 12, 13, 14, 15, 16, 17, 18, 19, 20, 21, 22, 23 and 24.
